(19) United States
(12) Patent Application Publication
(10) Pub. No.: US 2026/0306053 A1
(43) Pub. Date: Oct. 8, 2026

(54) METHOD AND SYSTEM FOR NONINVASIVE MAPPING OF ARRHYTHMIA USING ELECTROMECHANICAL WAVE IMAGING

(71) Applicant: THE TRUSTEES OF COLUMBIA UNIVERSITY IN THE CITY OF NEW YORK, New York, NY (US)

(72) Inventors: Elisa Konofagou, New York, NY (US); Maria Amalia Tourni, New York, NY (US)

(73) Assignee: THE TRUSTEES OF COLUMBIA UNIVERSITY IN THE CITY OF NEW YORK, New York, NY (US)

(21) Appl. No.: 19/641,976

(22) Filed: Apr. 8, 2026

Related U.S. Application Data
(60) Provisional application No. 63/785,345, filed on Apr. 8, 2025.

(51) Int. Cl. A61B 8/08; A61B 5/00; A61B 5/347; A61B 8/00

(52) U.S. Cl. CPC A61B 8/0883; A61B 5/347; A61B 5/725; A61B 8/461; A61B 8/485; A61B 8/5223

(57) ABSTRACT

The disclosed subject matter discloses systems and methods for generating a cardiac electromechanical activation map using ultrasound data acquired from a clinical ultrasound system operating at a frame rate less than 150 frames per second. The method includes acquiring ultrasound data of a heart of a subject and acquiring a corresponding electrocardiogram (ECG) signal, identifying an onset of electrical activation based on the ECG signal, processing the ultrasound data to obtain displacement information of myocardial tissue, calculating interframe strain based on the displacement information, determining activation time points based on zero-crossings of the interframe strain, and generating an electromechanical activation map indicating spatial propagation of activation across the myocardial tissue, thereby localizing at least one arrhythmic feature.

### CROSS-REFERENCE TO RELATED APPLICATION

This application is a non-provisional application, which claims priority to U.S. Provisional Patent Application No. 63/785,345, which was filed on Apr. 8, 2025, the entire content of which is incorporated by reference herein.

### GRANT INFORMATION

This invention was invention was made with government support under National Institutes of Health (NIH) Grant No. R01HL140646. The government has certain rights in the invention.

### BACKGROUND

Cardiac arrhythmias and other electrophysiological abnormalities can require accurate localization of electrical activation within the heart for effective diagnosis and treatment planning. In certain clinical practice, localization of arrhythmic sources prior to procedures such as catheter ablation is primarily performed using a 12-lead electrocardiogram (ECG). However, certain ECG processes provide limited spatial resolution and are insufficient to precisely identify the origin and propagation of abnormal electrical activity.

Electromechanical Wave Imaging (EWI) is a noninvasive ultrasound-based technique that can map cardiac electromechanical activation by detecting mechanical deformation of myocardial tissue associated with electrical activation. Certain EWI technologies require high frame rate ultrasound acquisition, typically on the order of thousands of frames per second, which necessitates specialized research-grade ultrasound systems that are not approved for routine clinical use. Further, certain EWI implementations can require access to raw radiofrequency (RF) ultrasound data, which is not generally available from certain clinical ultrasound systems that primarily output processed image data. These constraints can limit the use of EWI to research environments and prevent integration into standard clinical workflows.

Accordingly, there remains a need for an electromechanical activation mapping techniques using clinical ultrasound systems operating at clinically relevant frame rates, while maintaining sufficient accuracy for clinical diagnosis and treatment planning.

### SUMMARY

The disclosed subject matter provides techniques of electromechanical wave imaging (EWI) for activation map generation and arrhythmia localization.

In certain embodiments, the disclosed subject matter discloses systems and methods for generating an electromechanical activation map for a heart of a subject using ultrasound data acquired at a frame rate less than 150 frames per second. An example method includes acquiring ultrasound data of the heart and acquiring a corresponding electrocardiogram (ECG) signal, identifying an onset of electrical activation based on the ECG signal, obtaining displacement information of myocardial tissue in the heart from the ultrasound data, determining an interframe strain of the myocardial tissue based on the displacement information, determining one or more activation time points based on one or more zero-crossings of the interframe strain, and generating an electromechanical activation map indicating spatial propagation of activation across the myocardial tissue, thereby localizing at least one arrhythmic feature of the heart.

In certain embodiments, obtaining displacement information includes performing envelope detection using a Hilbert transform and applying logarithmic compression to generate B-mode images. In certain embodiments, the displacement information is obtained using a cross-correlation algorithm based on at least one of frame rate, imaging depth, or spatial resolution. In certain embodiments, determining the interframe strain includes applying a least-squares estimator, and the method further includes applying a temporal filter to the interframe strain, including a Gaussian filter.

In certain embodiments, the method further includes segmenting a myocardial region of the heart from the ultrasound data prior to determining the interframe strain. In certain embodiments, determining the activation time points includes applying a machine learning model trained to detect zero-crossings. The machine learning model includes a random forest classifier trained using higher frame rate ultrasound data. In certain embodiments, the machine learning classifier is trained to select the at least one zero-crossing by selecting a zero-crossing location with a highest probability for each pixel.

In certain embodiments, the electromechanical activation map includes one or more of a two-dimensional activation map or a three-dimensional activation map.

In certain embodiments, an example system for generating an electromechanical activation map for a heart of a subject, including an ultrasound acquisition device configured to acquire ultrasound data of the heart at a frame rate less than 150 frames per second, an ECG acquisition module configured to acquire an electrocardiogram signal of the subject, and a processing unit configured to identify an onset of electrical activation based on the ECG signal, process the ultrasound data to obtain displacement information of myocardial tissue of the heart, calculate an interframe strain based on the displacement information, determine one or more activation time points based on one or more zero-crossings of the interframe strain, and generate an electromechanical activation map representing spatial propagation of activation, thereby localizing at least one arrhythmic feature.

In certain embodiments, the ultrasound acquisition device comprises a portable ultrasound scanner. In certain embodiments, the processing unit is configured to generate a three-dimensional activation map from multiple ultrasound views.

The disclosed subject matter will be further described below.

### BRIEF DESCRIPTION OF THE DRAWINGS

The accompanying drawings are included to provide a further understanding of the disclosed subject matter. The drawings illustrate embodiments of the disclosure and, together with the description, serve to explain the principles of the disclosure.

FIG. 1 illustrates an example process for generating an electromechanical activation map, in accordance with certain embodiments of the disclosed subject matter.

FIG. 2 is a diagram to illustrate an example system for generating an electromechanical activation map, in accordance with certain embodiments of the disclosed subject matter.

FIG. 3A illustrates EWI activation map acquired from the standard apical 4-chamber view under epicardial pacing conditions, in accordance with certain embodiments of the disclosed subject matter.

FIG. 3B illustrates Corre-sponding epicardial electrical activation map con□rming earliest electrical activation at the same location with FIG. 3A, in accordance with certain embodiments of the disclosed subject matter.

FIG. 4A illustrates a full heart 2D EWI activation isochrones from four standard apical views, in accordance with certain embodiments of the disclosed subject matter.

FIG. 4B illustrates full heart 3D-rendered EWI activation isochrones generated from the coregistration of the four 2D views around the LV median axis in the posterior (left) and anterior (right) view, in accordance with certain embodiments of the disclosed subject matter.

FIG. 5A illustrates manually generated EWI 2D activation maps, in accordance with certain embodiments of the disclosed subject matter.

FIG. 5B illustrates RFC-based automated EWI maps, in accordance with certain embodiments of the disclosed subject matter.

FIG. 5C illustrates pre-ablation 12-lead ECG, in accordance with certain embodiments of the disclosed subject matter.

FIG. 5D illustrates post-ablation 12-lead ECG, in accordance with certain embodiments of the disclosed subject matter.

FIG. 6A illustrates manually generated EWI 2D activation maps and a three-dimensional activation map, in accordance with certain embodiments of the disclosed subject matter.

FIG. 6B illustrates RFC-based automated EWI maps, in accordance with certain embodiments of the disclosed subject matter.

FIG. 6C illustrates pre-ablation 12-lead ECG, in accordance with certain embodiments of the disclosed subject matter.

FIG. 6D illustrates post-ablation 12-lead ECG, in accordance with certain embodiments of the disclosed subject matter.

It is to be understood that both the foregoing general description and the following detailed description are exemplary and are intended to provide further explanation of the disclosed subject matter.

### DETAILED DESCRIPTION

The disclosed subject matter provides a clinical ultrasound system to identify where abnormal heart rhythms originate, without inserting any instruments into the body. Instead of directly measuring electrical signals inside the heart, the system observes how the heart muscle moves in response to those signals. By tracking precise, time-dependent changes in the motion of the heart muscle and analyzing when different regions begin to contract, the system determines the location and spread of the underlying electrical activation. The disclosed subject matter enables users to visualize the source and propagation of arrhythmias using routine ultrasound imaging, providing a fast, noninvasive alternative to traditional methods that rely on invasive procedures or specialized equipment.

For purposes of the disclosed subject matter, the following terms are used as described below. The definitions provided herein are intended to facilitate understanding of the disclosure and are not intended to be limiting. A person of ordinary skill in the art (POSITA) will recognize that variations, equivalents, and alternative interpretations consistent with the technical context are within the scope of the disclosed subject matter.

The terms “comprise(s),” “include(s),” “having,” “has,” “can,” “contain(s),” and variants thereof, as used herein, are intended to be open-ended transitional phrases, terms, or words that do not preclude additional acts or structures. The singular forms “a,” “an,” and “the” include plural references unless the context clearly dictates otherwise. The disclosed subject matter also contemplates other embodiments “comprising,” “consisting of,” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, e.g., with respect to biological systems or processes, the term can mean within an order of magnitude, within 5-fold, and within 2-fold, of a value.

As used herein, the term “coupled” refers to the connection of a device component to another device component by methods known in the art.

As used herein, the term “subject” includes any human or nonhuman animal. The term “nonhuman animal” includes, but is not limited to, all vertebrates, e.g., mammals and non-mammals, such as nonhuman primates, dogs, cats, sheep, horses, cows, chickens, amphibians, reptiles, etc.

As used herein, the term “interframe” refers to a relationship between two temporally consecutive frames in an ultrasound imaging sequence, wherein measurements are determined based on differences or correlations between the consecutive frames.

As used herein, the term “interframe strain” refers to a measure of deformation of myocardial tissue between consecutive frames, derived from displacement information, and indicative of local contraction or expansion of the tissue.

As used herein, the term “envelope detection” refers to a signal processing operation configured to extract an amplitude envelope of an ultrasound signal, including by applying a Hilbert transform.

As used herein, the term “onset of electrical activation” refers to a time point identified from an electrocardiogram (ECG) signal corresponding to an initiation of electrical activity within the heart, and serves as a temporal reference for aligning ultrasound data.

As used herein, the term “displacement” refers to motion of myocardial tissue between consecutive ultrasound frames, which is estimated using signal processing techniques including cross-correlation of ultrasound data.

As used herein, the term “zero-crossing” or “ZC” refers to a point in a strain signal at which the signal changes sign, corresponding to a transition associated with onset of mechanical contraction. As used herein, the term “zero-crossing selection” refers to identifying a time point corresponding to a zero-crossing in a strain signal for a given spatial location, wherein the time point represents an activation time of myocardial tissue, and is determined manually, semi-automatically, or automatically using a machine learning model.

As used herein, the term “activation time” refers to a time at which a region of myocardial tissue begins mechanical contraction in response to electrical activation, as determined from strain analysis.

As used herein, the term “isochrone map” refers to a spatial representation in which different regions of myocardial tissue are assigned activation times to visualize propagation of electromechanical activation.

As used herein, the term “frame rate” refers to a number of ultrasound image frames acquired per unit time.

As used herein, the term “machine learning model” refers to a computational model trained using data to perform a task including detecting zero-crossings in strain signals.

As used herein, the term “myocardial segmentation” refers to identifying and isolating myocardial tissue regions within ultrasound images for analysis.

Unless otherwise specified, each of the foregoing terms is not limited to a specific implementation, algorithm, or configuration, and encompasses variations that would be understood by a person of ordinary skill in the art in view of the disclosed subject matter.

In certain embodiments, the disclosed subject matter provides a system for electromechanical wave imaging (EWI) of a tissue of a subject. An example system can include a processing unit. The processing unit can be configured to perform the instructions specified by software stored in a hard drive, a removable storage medium, or any other storage media. The software can include computer codes, which can be written in a variety of languages, e.g., MATLAB and/or Microsoft Visual C++. Additionally or alternatively, the processing unit can include hardware logic, such as logic implemented in an application-specific integrated circuit (ASIC). The processing unit can be configured to control one or more of the system components. For example, and as embodied herein, the processing unit can be configured to control the operation of the transducer.

In certain embodiments, the processing unit can be configured to perform an automated selection of at least one zero-crossing (ZC) location. In non-limiting embodiments, the processing unit can be configured to perform the automated selection using a machine learning classifier. The machine learning classifier can be a logistic regression classifier, a support vector machine (SVM) classifier, a convolutional neuronal network (CNN) classifier, a recurrent neuronal network (RNN) classifier, or a Random Forest Classifier (RFC). In certain embodiments, the machine learning classifier can be a Random Forest classifier, which can include at least about 10, about 20, about 30, about 40, about 50, about 100, about 150, about 200, about 300, about 400, or about 500 estimators. In non-limiting embodiments, the machine learning classifier can be configured to have a precision rate of more than about 60%, about 70%, about 80%, about 90%, or about 95%.

In certain embodiments, the processing unit can be configured to collect a plurality of features from a dataset. The features can include a spatial coordinate, a positive to negative zero-crossing time-point, a slope of a strain curve at each zero-crossing location, a maximum positive strain value surrounding each zero-crossing location, a minimum negative strain value surrounding each zero-crossing location, a time-point location of the maximum and the minimum strain values, or combinations thereof. In non-limiting embodiments, the dataset can be EWI images, EWU strain curves, or a combination thereof. In certain embodiments, the dataset can be an external dataset and/or an internal dataset obtained through a transducer coupled to the disclosed processing unit. In some embodiments, additional features can be added to the dataset. For example, categorical features such as the apical view type where the strain curves are retrieved from (e.g., 4-chamber, 3.5-chamber, 2-chamber or 3-chamber view) can be included as well as the type of acquisition used (e.g., a field of view depth and pixel resolution).

In certain embodiments, the disclosed classifier can select ZC candidates based on the collected features. For example, the machine learning (ML) classifier can be a binary classifier. The ML classifier can vote for the best ZC candidate. For example, the ML classifier can select ZC location candidates with the highest probability for each pixel and assign it with a label (e.g., 1). The ML classifier can swipe through a set of probability thresholds to find a cutoff value (e.g., highest precision given a condition set on the recall, also known as sensitivity or true positive rate). Then, the ML classifier can pick a ZC candidate if its probability is higher than the cutoff threshold and assign it the label (e.g., 1). The probability thresholds can range from 0 to 1, less likely candidates to most likely. For example, candidates with probabilities lying above thresholds set to 0.7, 0.8, or 0.9 can be considered as satisfying cut off values and selected. These candidates can be considered to have a high enough probability, sufficient to be assigned a label 1, while the others can be discarded and assigned a label 0.

In certain embodiments, the machine learning classifier can be trained with a training dataset. In non-limiting embodiments, the training dataset can include hyperparameters. For example, the logistic regression model can include hyperparameters (e.g., elastic net, L1-ration, etc.), the SVM model can include hyperparameters (e.g., kernel types), and the Random Forest model can include hyperparameters (e.g., n estimators). The machine learning classifier can be fitted on the training set with a set of given hyperparameters and evaluated its performance. In some embodiments, the training dataset can include both training and validation samples for improving the accuracy and efficiency of the machine learning classifier.

For example, once the machine learning classifier and a set of hyperparameters that can yield appropriate results on the validation set are determined, the classifier can be refitted with these settings on the dataset constituted of both the training and validation samples for improving the ML model. In non-limiting embodiments, the training dataset can be ground truth samples to automatically select ZCx. The ground truth samples are manually generated isochrones. Isochrones can be the electromechanical wave imaging activation maps. In certain embodiments, the processing unit can be configured to automatically select using a heuristic-based baseline. In non-limiting embodiments, the Heuristic-based baseline can be an approach relying on certain hard coded values and pre-set conditions. For example, the heuristic-based baseline can select the first positive to negative ZC immediately following the QRS complex onset of the obtained incremental axial strain curve and occurring within a pre-determined search window (e.g., 200 ms). In non-limiting embodiments, the heuristic-based baseline can select the first positive to negative ZC that satisfies the pre-determined empirical conditions.

In certain embodiments, the system can be configured to perform electromechanical imaging. The disclosed system can further include a transducer that can be configured to perform electromechanical wave imaging. For example, EWI images of a subject can be acquired through the disclosed transducer, which can generate a high frame-rate ultrasound sequence (e.g., up to about 2 seconds, up to about 4 seconds, up to about 6 seconds, or up to about 8 seconds) including a single diverging wave (e.g., at about 250 Hz, at about 500 Hz, at about 1000 Hz, or at about 2000 Hz, or between about 205 Hz to about 2000 Hz) acquired in four standard echocardiographic apical views. In non-limiting embodiments, the transducer can be a 2.5 MHz phased-array transducer. In some embodiments, the sequences can have a duration of about 0.5-2.5 seconds, framerates of about 250 Hz-4000 Hz and the transducer frequencies can vary from about 2 to 4 MHz. The processing unit can perform the displacement estimation on the radiofrequency signals from each element of the ultrasound probe/transducer with 1D axial cross-correlation (e.g., 10 wavelength window, 90% overlap). The processing unit can further calculate the axial strains from the displacement estimation by a least-squares estimator. The processing unit can be operatively coupled to the transducer. For example, the processing unit and the transducer can be coupled directly (e.g., wire connection or installation into the transducer) or indirectly (e.g., wireless connection).

In certain embodiments, the disclosed processing unit can identify multiple ZC candidates. In the apical views, since the ultrasound beam can be aligned with the ventricular myocardial wall that is shortening during systole, this corresponds to a positive-to-negative flip or downward ZC of the incremental axial strain. As incremental strain curves sometimes exhibit more than one negative peak after the onset of the QRS complex or p-wave (e.g., for the ventricular or atrial isochrones, respectively), instead of one clear ZC, multiple ZC candidates can be identified.

In certain embodiments, the disclosed processing unit can be configured to generate an activation map/EWI isochrone. The disclosed pocessing unit can be configured to perform beamforming on an image acquired by the transducer, estimate the displacement of the tissue based on the image, and generate an incremental strain curve based on the displacement. For example, the myocardium of interest can be first segmented on the B-mode image. For about 150 randomly chosen points in the segmented mask, t the timing of the first significant change after the QRS onset can be selected manually/automatically on the incremental axial strain curves for the ventricles, respectively p-wave onset.

A Delaunay triangulation-based cubic interpolation can be applied to the 2D scattered activation time ZC values in order to achieve a continuous isochrone pattern throughout the entire myocardium mask grid. The activation timings in milliseconds can be color-coded on a 2D map or isochrone (e.g., with red being early activation and blue late). Once the maps have been generated in the four views, a 3D-rendering algorithm can automatically detect the longitudinal left ventricular (LV) median axis on the four apical isochrones. The multi-2D views can then be co-registered around that axis, and linear interpolation of the activation times can be performed around the circumference. This provides 3D-rendered activation maps.

The machine learning classifier and/or heuristic-based baseline can reduce the time for generating the activation map/EWI isochrone, address the cases with multiple ZCs, and choose a suitable candidate consistently without observer bias. In non-limiting embodiments, the disclosed algorithm can perform the automated selection in less than about 30 seconds, about 20 seconds, about 10 seconds, about 5 seconds, about 3 seconds, about 1 seconds per isochrone. In some embodiments, if the algorithm is run with a given set of input parameters and strain curves, the disclosed algorithm can be configured to always give out the same results. Such an algorithm can reduce the inter-observer variability, which can occur when more than two different human operators manually generate the isochrones (e.g., observer's bias).

In certain embodiments, the subject can include a Wolff-Parkinson-White (WPW) patient, a healthy human, any animal models (e.g., canines), any patients after successful ablation procedures, an Atrial Flutter patient, a ventricular trachycardia patient, or combinations thereof. In non-limiting embodiments, the disclosed model can be applied to any type of subjects and/or patients, as long as the training dataset can be updated in the meantime, and the model can be retrained to include such type of data.

In certain embodiments, the disclosed subject matter provides a method for electromechanical wave imaging (EWI) with automated activation map generation. An example method can include performing an automated selection of at least one zero-crossing location using a heuristic-based baseline and/or a machine learning classifier and generating an electromechanical wave imaging isochrone based on the automated selection.

In certain embodiments, through the heuristic-based baseline, the first positive to negative ZC immediately following the QRS complex onset of the obtained incremental axial strain curve and occurring within a pre-determined search window (e.g., 200 ms) can be selected. In non-limiting embodiments, through the heuristic-based baseline, the first positive to negative ZC that satisfies the pre-determined empirical conditions can be selected. The machine learning classifier can be trained to select the at least one zero-crossing by selecting a zero-crossing location with a highest probability for each pixel, or selecting a zero-cross location that has a probability higher than a cutoff threshold, where-in the cutoff threshold is determined based on a sensitivity and/or true positive value of the dataset.

In certain embodiments, the automated ZC selection can be performed using a machine learning classifier. The machine learning classifier can be a logistic regression classifier, a support vector machine (SVM) classifier, or a Random Forest classifier. In some embodiments, the machine learning classifier can be a Random Forest classifier, which can include at least about 10, about 20, about 30, about 40, about 50, about 100, about 150, about 200, about 300, about 400, or about 500 estimators. In non-limiting embodiments, the machine learning classifier can be configured to have a precision rate of more than about 60%, about 70%, about 80, about 90%, or about 95%.

In certain embodiments, the method can further include collecting a plurality of features from a dataset. The features can include a spatial coordinate, a positive to negative zero-crossing time-point, a slope of a strain curve at each zero-crossing location, a maximum positive strain value surrounding each zero-crossing location, a minimum negative strain value surrounding each zero-crossing location, a time-point location of the maximum and the minimum strain values, or combinations thereof. In non-limiting embodiments, the disclosed classifier can select ZC candidates based on the collected features. For example, the ML classifier can vote for the best ZC candidate based on the collected features. The ML classifier can select ZC location candidates with the highest probability for each pixel and assign it with a label (e.g., 1). The ML classifier can swipe through a set of probability thresholds to find the best cutoff value (e.g., highest precision given a condition set on the recall, also known as sensitivity or true positive rate). Then, the ML classifier can pick the best ZC candidate if its probability is higher than the cutoff threshold and assign it the label (e.g., 1).

In certain embodiments, the dataset can be EWI images, EWU strain curves, or a combination thereof. In some embodiments, the dataset can be an external dataset and/or an internal dataset obtained through a transducer coupled to the disclosed processing unit. In some embodiments, additional features can be added to the dataset. For example, categorical features such as the apical view type where the strain curves are retrieved from (e.g., 4-chamber, 3.5-chamber, 2-chamber or 3-chamber view) can be included as well as the type of acquisition used (e.g., a field of view depth and pixel resolution).

In certain embodiments, the method can further include training the machine learning classifier with a training dataset. In non-limiting embodiments, the training dataset can include hyperparameters. For example, the machine learning classifier can be fitted on the training set with a set of given hyperparameters and evaluated its performance. In some embodiments, the training dataset can include both training and validation samples for improving the accuracy and efficiency of the machine learning classifier.

In non-limiting embodiments, the training dataset can be ground truth samples to automatically select ZCx. The ground truth samples are manually generated isochrones. In some embodiments, the machine learning classifier can be trained to select at least one ZC by selecting a zero-crossing location with the highest probability for each pixel or selecting a zero-cross location that has a probability higher than a cutoff threshold. The cutoff threshold can be determined based on the sensitivity and/or true positive value of a dataset. By looking at precision and recall curves, a user can choose a satisfying cutoff threshold depending on its requirements for a given application. In non-limting embodiments, a user can chose a threshold to favor high precision over high recall and vice versa. In some embodiments, the chosen cutoff value can be subjective and depends on the type of data on which the algorithm is being run.

### Example 1

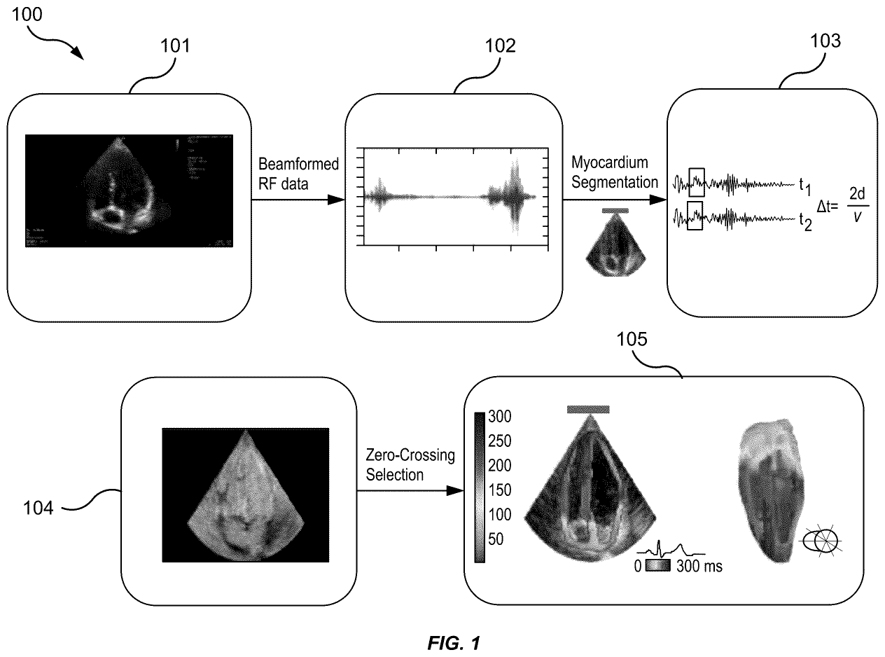

Referring to FIG. 1, an example EWI method 100 for generating a cardiac electromechanical activation map, can include at least data acquisition, interframe, axial strain generation, and isochrone generation via zero-crossing (ZC) selection.

As shown in FIG. 1, at 101), acquiring ultrasound data of a heart of a subject using a clinical ultrasound system can be operated at a frame rate of about 50-100 frames per second, thereby acquiring a corresponding electrocardiogram (ECG) signal of the subject. The ultrasound data include beamformed radiofrequency (RF) data obtained using a focused transmit sequence with a phased array probe at an imaging depth of about 10-20 cm. The ECG signal can be used to identify an onset of electrical activation afterwards, including identifying a P-wave onset for atrial activation or a QRS complex onset for ventricular activation, and selecting a corresponding starting frame of the ultrasound data.

At 102), the acquired ultrasound data are processed by performing envelope detection using a Hilbert transform to extract an analytic signal, followed by applying logarithmic compression to generate B-mode image data. A myocardial region of interest is defined using manual segmentation of the myocardium, and the processed data are limited to the segmented myocardial region for further analysis.

At 103), a displacement of myocardial tissue is determined using a one-dimensional radiofrequency cross-correlation algorithm, where parameters including window size and window shift can be dynamically adjusted based on acquisition parameters including frame rate, imaging depth, and pixel resolution.

The displacement information can be used to calculate interframe strain at 104) using a least-squares estimator with a kernel size of about 5 mm, followed by applying a temporal Gaussian filter having a kernel size of about 8 to smooth the strain data.

At 105), activation time points are determined by identifying zero-crossings in the interframe strain signals. A subset of spatial points across the myocardial region is selected, and corresponding strain curves are analyzed to identify a downward zero-crossing indicative of onset of mechanical contraction. The zero-crossing selection can be performed using a machine learning model, including a random forest classifier trained using higher frame rate ultrasound datasets. The machine learning model can predict zero-crossing time points for the selected spatial points based on features extracted from the strain signals. The machine learning model can employ a machine learning classifier, selected from at least one of a logistic regression classifier, a support vector machine (SVM) classifier, a convolutional neuronal network (CNN) classifier, a recurrent neuronal network (RNN) classifier, or a Random Forest classifier (RFC). In certain embodiments, the machine learning classifier can be a Random Forest classifier (RFC), which can include at least about 10, about 20, about 30, about 40, about 50, about 100, about 150, about 200, about 300, about 400, or about 500 estimators.

Activation times for remaining spatial locations are determined using interpolation across the myocardial region to generate a continuous activation profile. Finally, at 5), a two-dimensional and three-dimensional electromechanical activation map is generated by assigning activation times to corresponding spatial locations, thereby visualizing propagation of electromechanical activation from earliest to latest regions.

In certain embodiments, multiple two-dimensional activation maps obtained from different imaging views, including apical 4-chamber, 3-chamber, 2-chamber, and 3.5-chamber views, are combined to generate a three-dimensional activation map, providing a volumetric representation of activation propagation across cardiac chambers.

The generated activation map enables identification of at least one arrhythmic feature, including a location of earliest activation corresponding to an arrhythmic source and a propagation pattern indicative of normal or abnormal conduction pathways. The method is performed using a clinical ultrasound system without requiring high-frame-rate acquisition hardware, thereby enabling noninvasive, rapid, and clinically deployable electromechanical activation mapping.

### Example 2

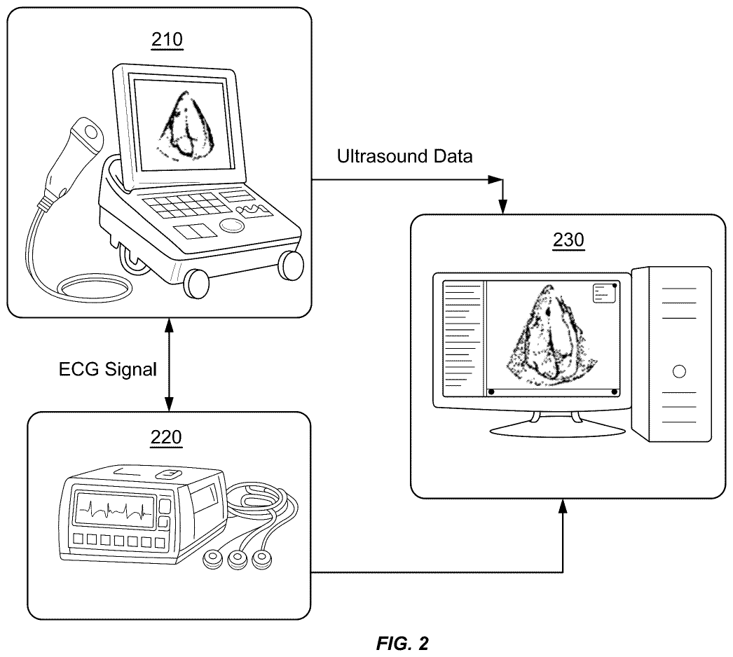

Referring to FIG. 2, an example system 200 includes an ultrasound acquisition device 210, an electrocardiogram (ECG) acquisition module 220, a processing unit 230. Such the above components are communicatively coupled.

The ultrasound acquisition device 210 is configured to acquire ultrasound data of a heart of a subject at a frame rate less than 150 frames per second, including by operating under control of one or more imaging parameters that set a transmit sequence, imaging depth, and frame rate. In certain embodiments, the ultrasound acquisition device 210 includes a portable ultrasound scanner having a phased array probe configured to perform cardiac imaging using a focused transmit sequence. The ultrasound acquisition device 210 generates beamformed radiofrequency (RF) data and corresponding image data.

The ECG acquisition module 220 is configured to acquire an electrocardiogram signal of the subject and provide timing information corresponding to electrical activity of the heart, including identification of P-wave onset or QRS complex onset.

The processing unit 230 is configured to receive the ultrasound data and the ECG signal. The processing unit 230 identifies an onset of electrical activation based on the ECG signal and synchronizes the ultrasound data with the identified onset.

The processing unit 230 processes the ultrasound data to obtain displacement information of myocardial tissue. In one implementation, the processing unit 230 performs envelope detection using a Hilbert transform and applies logarithmic compression to generate B-mode data.

The processing unit 230 determines displacement using a cross-correlation algorithm, wherein parameters including window size and window shift are dynamically adjusted based on frame rate, imaging depth, and spatial resolution. Further, the processing unit 230 calculates interframe strain based on the displacement using a least-squares estimator and applies a temporal filter including a Gaussian filter to the interframe strain. The processing unit 230 determines activation time points based on zero-crossings of the interframe strain. The zero-crossings are identified using a machine learning model including a random forest classifier trained on higher frame rate data.

The processing unit 230 generates an electromechanical activation map representing spatial propagation of activation across myocardial tissue and identifies at least one arrhythmic feature including a location of earliest activation.

In certain embodiments, the processing unit 230 can further include a display unit for visualizing the electromechanical activation mapping. The display unit is configured to receive the electromechanical activation map from the processing unit 230 and display the activation map. The display unit 240 presents at least one of a two-dimensional activation map or a three-dimensional activation map, wherein activation timing is visually represented across myocardial regions.

### Example 3

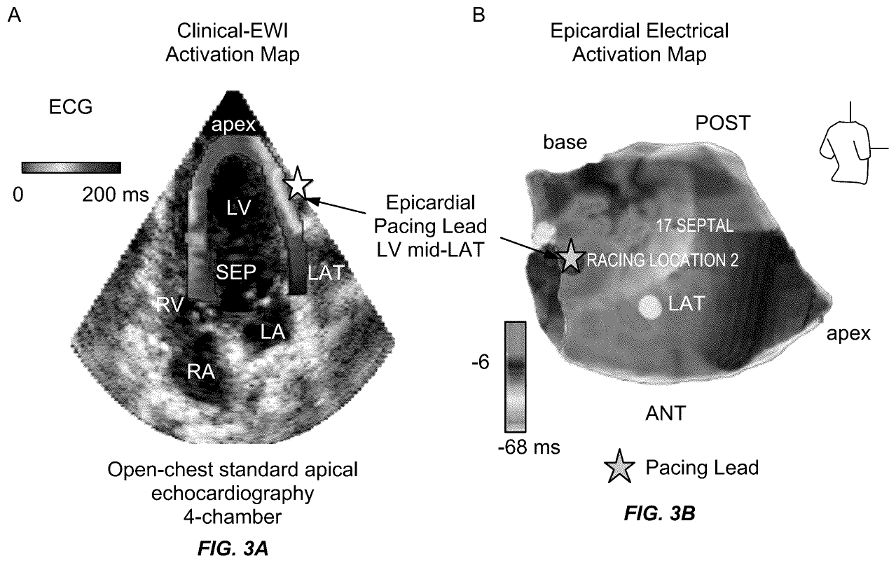

FIGS. 3A-3B illustrate an example validation of electromechanical activation mapping under controlled cardiac pacing conditions. Ultrasound data of a heart are acquired from a subject under paced conditions using a clinical ultrasound system operating at a frame rate of about 100 frames per second. The ultrasound data are obtained from a standard apical 4-chamber view at an imaging depth of about 8 cm using a phased array probe. Simultaneously, an electrocardiogram (ECG) signal is acquired to provide temporal reference information.

The heart is subjected to controlled electrical pacing using an epicardial pacing lead positioned at a mid-lateral wall of a left ventricle. The pacing is performed at a cycle length of about 400 ms, thereby establishing a known electrical activation origin within the myocardial tissue.

The ultrasound data are processed to determine displacement and interframe strain of myocardial tissue, and activation time points are determined based on zero-crossings of the interframe strain, as described in Example 1. An electromechanical activation map is generated representing spatial propagation of activation across the myocardial tissue.

As illustrated in FIG. 3A, the electromechanical activation map identifies an earliest activation region corresponding to the pacing location at the mid-lateral wall of the left ventricle (LV). The earliest activation region is visually indicated and is distinguished from later activation regions based on relative activation timing. Red corresponds to the earliest activation site, while blue to the latest.

A corresponding electrical activation map is obtained using an invasive electroanatomic mapping system under the same pacing conditions. As illustrated in FIG. 3B, the electrical activation map identifies the earliest electrical activation at the same mid-lateral left ventricular location.

Comparison between the electromechanical activation map and the electrical activation map demonstrates spatial correspondence between the earliest activation regions, thereby validating that the electromechanical mapping accurately identifies the origin of activation under controlled conditions.

Further, the electromechanical activation map shows propagation of activation from the mid-lateral wall toward septal and apical regions of the heart, consistent with expected physiological propagation patterns under paced conditions.

This example demonstrates that electromechanical activation mapping performed using clinical ultrasound data at low frame rates accurately localizes activation origin and propagation without requiring invasive measurement or high-frame-rate imaging systems.

### Example 4

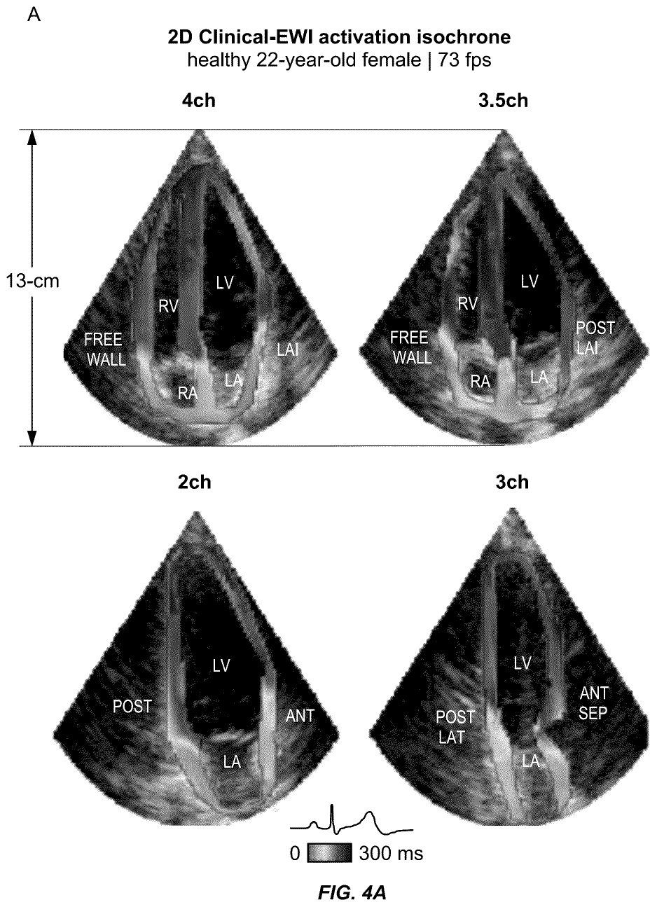

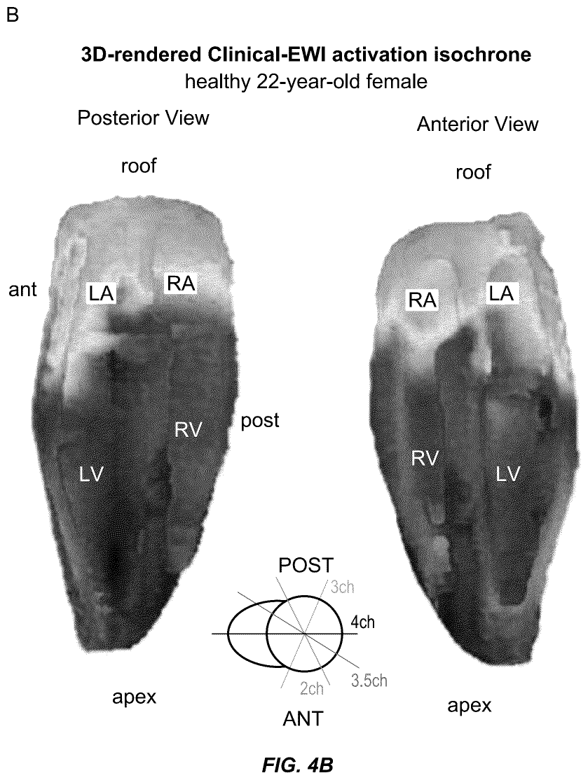

FIGS. 4A-4B illustrate example EWI activation mapping using a portable clinical scanner. The electromechanical activation in a healthy subject is determined under physiological conditions. Ultrasound data of a heart of a subject are acquired using a clinical ultrasound system at a frame rate of about 73 frames per second, with an imaging depth of about 13 cm and a field of view of about 90 degrees. The ultrasound data are obtained from multiple apical views, including apical 4-chamber, 3.5-chamber, 2-chamber, and 3-chamber views. A corresponding electrocardiogram (ECG) signal is acquired to provide temporal reference information.

The ultrasound data are processed to determine displacement and interframe strain of myocardial tissue, and activation time points are determined based on zero-crossings of the interframe strain, as described in Example 1. Two-dimensional electromechanical activation maps are generated for each of the apical views, representing spatial propagation of activation across myocardial tissue.

As illustrated in FIG. 4A, the electromechanical activation maps show earliest activation originating in a right atrial region. The activation propagates through an atrioventricular junction and across an interventricular septum, followed by propagation through ventricular regions toward an apex of the heart. Early activation is represented in red, while later activation is shown in blue.

The activation pattern exhibits a temporally consistent sequence across the multiple views, indicating coordinated and symmetric propagation across myocardial walls. The latest activation is observed at the apical region, consistent with expected physiological activation patterns in a healthy heart.

The two-dimensional activation maps from the multiple views are combined to generate a three-dimensional activation map, as illustrated in FIG. 4B. Both posterior and anterior views of the 3D map further illustrate the physiological propagation pattern across all four cardiac chambers. The three-dimensional activation map provides a volumetric representation of activation propagation across cardiac chambers, including atrial and ventricular regions.

The electromechanical activation mapping demonstrates a physiological activation sequence originating from the atrial region and propagating through the ventricular myocardium in a coordinated manner. This example demonstrates that electromechanical activation mapping using clinical ultrasound data at low frame rates can accurately capture normal cardiac activation patterns in a noninvasive manner.

### Example 5

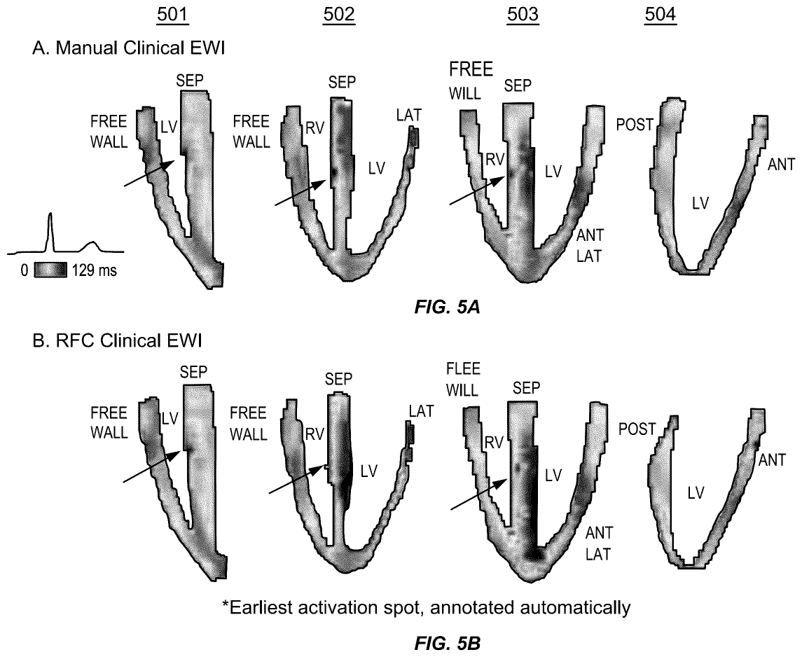

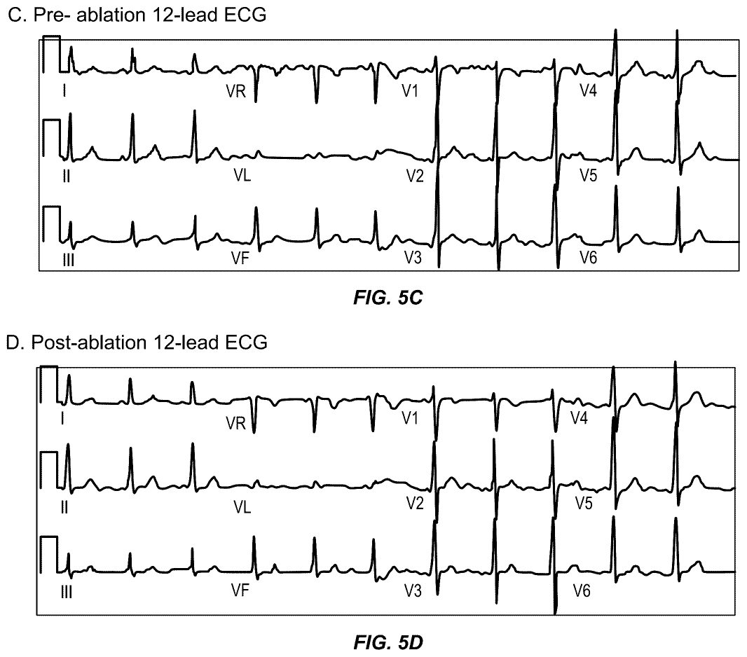

Referring to FIGS. 5A-5D, an example electromechanical activation map in a subject diagnosed with an arrhythmic condition is presented. Ultrasound data of a heart of a subject are acquired using a clinical ultrasound system at a frame rate of about 68 frames per second, with an imaging depth of about 14 cm and a field of view of about 90 degrees. The ultrasound data are obtained from multiple apical views, including apical 4-chamber (501 in FIG. 5A-5B), 5-chamber (501 in FIG. 5A-5B), 3.5-chamber (503 in FIG. 5A-5B), and 2-chamber (504 in FIG. 5A-5B) views. A corresponding electrocardiogram (ECG) signal is acquired to provide temporal reference information.

The subject undergoes an electrophysiological evaluation indicating the presence of an accessory conduction pathway. The ultrasound data are processed to determine displacement and interframe strain of myocardial tissue, and activation time points are determined based on zero-crossings of the interframe strain, as described in Example 1. Two-dimensional electromechanical activation maps are generated for each of the apical views.

As illustrated in FIG. 5A, the electromechanical activation maps identify an earliest activation region localized to a mid-septal right-sided wall of the heart. The activation propagates from the mid-septal region toward adjacent myocardial regions, including anterior and lateral regions, indicating a non-physiological activation pattern.

The activation pattern corresponds to a fascicular ventricular accessory pathway, characterized by conduction that bypasses a normal atrioventricular nodal pathway. The earliest activation location identified by the electromechanical activation map is consistent across multiple imaging views.

Automated activation maps generated using a machine learning model, as illustrated in FIG. 5B, identify the same earliest activation region as manually generated activation maps, demonstrating consistency between automated and manual processing.

In certain embodiments shown in FIG. 5B, random forest classifier (RFC) can be used in the machine learning model comprising a plurality of decision trees configured to perform classification based on input features derived from data, wherein the model aggregates outputs from the plurality of decision trees to generate a predicted classification result. Unless otherwise specified, the RFC machine learning algorithm is not limited to a specific number of trees, training method, or feature set, and encompasses variations that would be understood by a person of ordinary skill in the art in view of the disclosed subject matter. The predicted earliest activation site aligns with the manually annotated maps. Pre-excitation delta waves remained visible in the post-ablation 12-lead ECG (FIGS. 5C-5D).

The electromechanical activation results are compared with findings from an invasive electrophysiological study, which confirms the presence and location of the accessory pathway at the mid-septal region. The electromechanical activation mapping correctly identifies the location of the accessory pathway without requiring invasive procedures. This example demonstrates that electromechanical activation mapping using clinical ultrasound data at low frame rates can accurately localize abnormal conduction pathways and provide diagnostic information beyond that available from surface electrocardiography.

### Example 6

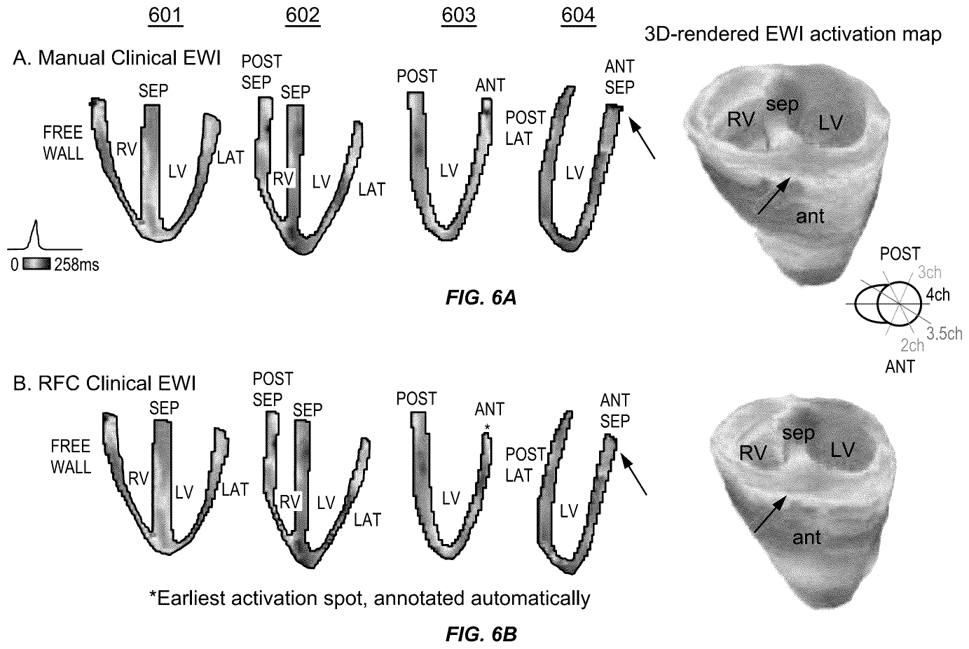

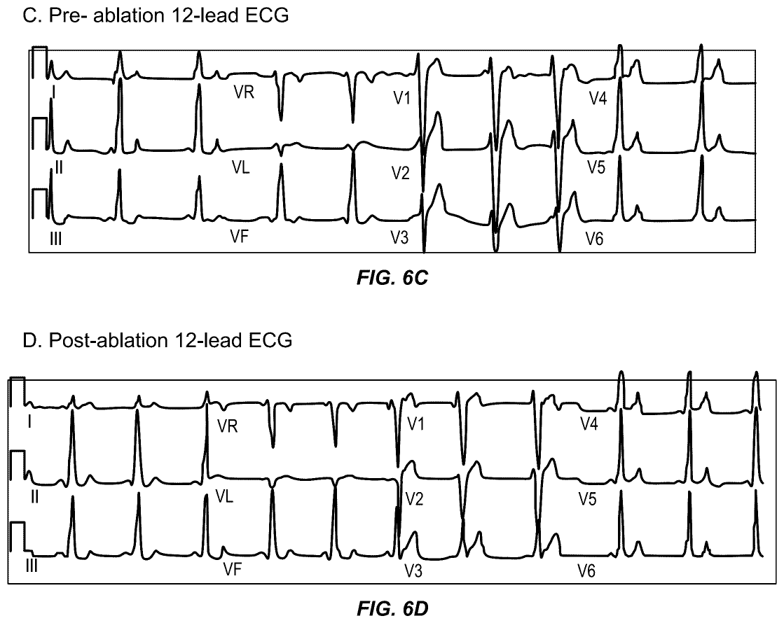

Referring to FIGS. 6A-6D, the EWI process is performed to determine electromechanical activation in a subject diagnosed with Wolff-Parkinson-White (WPW) syndrome. Ultrasound data of a heart of a subject are acquired using a clinical ultrasound system at a frame rate of about 61 frames per second, with an imaging depth of about 16 cm and a field of view of about 90 degrees. The ultrasound data are obtained from multiple apical views, including apical 4-chamber (601 in FIG. 6A-6B), 3.5-chamber (602 in FIG. 6A-6B), 2-chamber (603 in FIG. 6A-6B), and 3-chamber (604 in FIG. 6A-6B) views. A corresponding electrocardiogram (ECG) signal is acquired to provide temporal reference information.

The ultrasound data are processed to determine displacement and interframe strain of myocardial tissue, and activation time points are determined based on zero-crossings of the interframe strain, as described in Example 1. Two-dimensional electromechanical activation maps are generated for each of the apical views.

As illustrated in FIG. 6A, manually generated electromechanical activation maps identify an earliest activation region localized to an anteroseptal basal region of the left ventricle. The activation propagates from the anteroseptal region toward adjacent myocardial regions, including posterior and lateral regions, indicating a non-physiological activation pattern associated with an accessory conduction pathway.

As illustrated in FIG. 6B, automated activation maps generated using a machine learning model, including a random forest classifier, identify the same earliest activation region. The automated activation maps show agreement with manually generated maps and preserve overall propagation patterns across myocardial tissue.

The two-dimensional activation maps are combined to generate a three-dimensional activation map, as further illustrated in FIG. 6A and FIG. 6B, providing a volumetric representation of activation propagation across cardiac chambers. The three-dimensional activation map identifies the earliest activation region at the anteroseptal basal wall.

As illustrated in FIG. 6C, a pre-ablation electrocardiogram (ECG) signal exhibits a pre-excitation pattern suggestive of an anteroseptal accessory pathway. As illustrated in FIG. 6D, a post-ablation electrocardiogram (ECG) signal is obtained for comparison. Pre-procedural 12-lead ECG clinical diagnosis predicts an anteroseptal AP (FIG. 6C). Post-ablation 12-lead ECG maintains pre-ablation morphology, as the procedural intervention is avoided (FIG. 6D).

An invasive electrophysiological study identifies an accessory pathway located at or near a His-bundle region. Due to proximity to a normal conduction system, the accessory pathway is not ablated. The electromechanical activation mapping identifies the earliest activation region corresponding to the accessory pathway location.

This example demonstrates that electromechanical activation mapping using clinical ultrasound data at low frame rates identifies accessory pathway locations consistent with electrophysiological mapping, including cases in which ablation is not performed due to anatomical constraints.

As noted in the above examples, the disclosed systems and methods demonstrate that electromechanical activation mapping can be accurately performed using clinical ultrasound systems operating at reduced frame rates. Validation across controlled pacing conditions, healthy physiological conditions, and subjects with arrhythmic disorders shows that the generated activation maps consistently localize earliest activation regions in agreement with invasive electrical mapping and clinical diagnoses. The processing pipeline, including envelope detection, adaptive displacement estimation, and strain-based activation detection, enables reliable extraction of electromechanical signals despite reduced temporal resolution, while machine learning-based zero-crossing detection improves consistency and reduces operator dependence.

The disclosed approach further enables rapid and noninvasive mapping of cardiac activation with processing times suitable for clinical workflows, including pre-procedural planning and real-time decision support. The system operates using standard, portable ultrasound hardware without requiring specialized acquisition systems, enabling scalable deployment. While reduced frame rate introduces limitations in absolute timing precision, relative activation patterns remain robust for identifying arrhythmic features. The combination of automation, portability, and compatibility with existing clinical systems supports integration into routine cardiac assessment and expands accessibility of electromechanical mapping.

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. The materials, methods, and examples disclosed herein are illustrative only and not intended to be limiting.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

What is claimed is:

1. A method for generating an electromechanical activation map for a heart of a subject, comprising:
acquiring ultrasound data of the heart at a frame rate of less than 150 frames per second;
acquiring an electrocardiogram (ECG) signal of the subject;
identifying an onset of electrical activation based on the ECG signal;
obtaining displacement information of myocardial tissue in the heart from the ultrasound data;
determining, using a processing unit, an interframe strain of the myocardial tissue based on the displacement information;
determining, using the processing unit, one or more activation time points based on one or more zero-crossings of the interframe strain; and
generating an electromechanical activation map indicating spatial propagation of activation across the myocardial tissue, thereby localizing at least one arrhythmic feature of the heart.

2. The method of claim 1, wherein the obtaining displacement information comprises performing envelope detection using a Hilbert transform.

3. The method of claim 1, wherein the obtaining displacement information further comprises applying logarithmic compression to generate B-mode images.

4. The method of claim 1, wherein the displacement information is obtained using a cross-correlation algorithm based on at least one of frame rate, imaging depth, or spatial resolution.

5. The method of claim 1, wherein the determining the interframe strain comprises applying a least-squares estimator.

6. The method of claim 1, further comprising applying a temporal filter to the interframe strain.

7. The method of claim 6, wherein the temporal filter comprises a Gaussian filter.

8. The method of claim 1, further comprising segmenting a myocardial region of the heart from the ultrasound data prior to determining the interframe strain.

9. The method of claim 1, wherein the determining the activation time points comprises applying a machine learning model trained to detect zero-crossings.

10. The method of claim 9, wherein the machine learning model comprises a random forest classifier trained using higher frame rate ultrasound data.

11. The method of claim 10, wherein the machine learning classifier is trained to select the at least one zero-crossing by selecting a zero-crossing location with a highest probability for each pixel.

12. The method of claim 1, wherein the electromechanical activation map comprises one or more of a two-dimensional activation map or a tree-dimensional activation map.

13. A system for generating an electromechanical activation map for a heart of a subject, comprising:
an ultrasound acquisition device configured to acquire ultrasound data of the heart at a frame rate less than 150 frames per second;
an ECG acquisition module configured to acquire an electrocardiogram signal of the subject; and
a processing unit configured to:
identify an onset of electrical activation based on the ECG signal;
process the ultrasound data to obtain displacement information of myocardial tissue of the heart;
calculate an interframe strain based on the displacement information;
determine one or more activation time points based on one or more zero-crossings of the interframe strain; and
generate an electromechanical activation map representing spatial propagation of activation, thereby localizing at least one arrhythmic feature.

14. The system of claim 13, wherein the ultrasound acquisition device comprises a portable ultrasound scanner.
The system of claim 13, wherein the processing unit is configured to generate a three-dimensional activation map from multiple ultrasound views.
